(19) United States
(12) Patent Application Publication
(10) Pub. No.: US 2026/0124267 A1
(43) Pub. Date: May 7, 2026

(54) COMPOSITIONS AND METHODS FOR IMPROVING BENEFIT OF AN EXERCISE

(71) Applicant: NANJING NUTRABUILDING BIO-TECH CO., LTD., Nanjing (CN)

(72) Inventors: Dawei JIN, Nanjing (CN); Kylin LIAO, Nanjing (CN); Shawn WELLS, Frisco, TX (US)

(21) Appl. No.: 19/397,425

(22) Filed: Nov. 21, 2025

Related U.S. Application Data
(63) Continuation of application No. PCT/CN2024/094882, filed on May 23, 2024.

(30) Foreign Application Priority Data
May 24, 2023 (WO) PCT/CN2023/095935

(51) Int. Cl. A61K 36/906; A61P 3/04; A61P 3/06; A61P 3/10

(52) U.S. Cl. CPC A61K 36/906; A61P 3/04; A61P 3/06; A61P 3/10

(57) ABSTRACT

The present invention provides methods for improving a benefit of an exercise experienced by a subject, comprising administration to the subject a composition comprising an effective amount of Grains of Paradise, analog or derivative thereof.

### CROSS REFERENCE TO RELATED APPLICATION

This application is a continuation application of International Patent Application No. PCT/CN2024/094882, filed on May 23, 2024, which claims the priority of the International Patent Application No. PCT/CN2023/095935, filed on May 24, 2023, the contents of all of which are incorporated herein by reference in their entirety.

### FIELD OF THE INVENTION

This invention generally relates to compositions and methods for improving a benefit of an exercise experienced by a subject, comprising administration to the subject a composition comprising an effective amount of Grains of Paradise, analog or derivative thereof.

### BACKGROUND OF THE INVENTION

Metabolic dysfunction (like obese, etc) is an important risk factor for life threatening diseases that can target cardiovascular, metabolic and hepatic systems. Nowadays, the prevalence of metabolic dysfunction is rapidly increasing in developed and developing countries, leading to increased morbidity and mortality due to type 2 diabetes mellitus, non-alcoholic fatty liver disease, and cardiovascular disease (Abdelaal, M., C. W. le Roux, and N. G. Docherty, Morbidity and mortality associated with obesity. Annals of translational medicine, 2017. 5(7): p. 161.).

Obesity and type 2 diabetes present a serious health concern globally. An imbalance in energy intake and expenditure results in weight gain and obesity. Mammals possess two specialized types of adipose tissues that serve opposite functions, white and brown adipose tissues (WAT and BAT for short respectively). Excess weight is stored in the form of lipids in white adipose tissue (WAT). In addition to these fat-storing cells, brown adipocytes also exist which can transform energy into heat, a process known as thermogenesis. Activation of thermogenesis by brown adipose tissue (BAT) can substantially contribute to weight loss and glucose metabolism.

Grains of Paradise (Aframomum melegueta, GP)—also known as alligator pepper or Guinea pepper-is an ancient spice from the ginger family (Zingiberaceae). Native to West Africa, its seeds, with their ginger-type bite, have traditionally been used to flavor foods (as a replacement for pepper), and more recently, as a flavoring in craft beer. It also historically been used in traditional African medicine for a wide range of ethnobotanical applications, such as treating stomachache, diarrhea and snake bites. Additionally, there are reported studies on antiulcer, cytoprotective and antimicrobial activities as well as sexual performance enhancing effects of Grains of Paradise.

In this invention, we found a unique composition including Grains of Paradise that could improve a benefit of an exercise regimen and promote the “browning” of white adipose tissue which could enhance weight loss.

### SUMMARY OF THE INVENTION

This summary is provided to introduce a selection of concepts in a simplified form that is further described below in the Detailed Description. This summary is not intended to identify key features or essential features of the claimed subject matter, nor is it intended to be used to limit the scope of the claimed subject matter.

One aspect of this invention relates to a method for improving a benefit of an exercise experienced by a subject, comprising administration to the subject a composition comprising an effective amount of Grains of Paradise, analog or derivative thereof.

In some embodiments, the benefit comprises increasing energy expenditure, reducing a body fat percentage, reducing weight, lowering a blood glucose, decreasing a blood triglyceride level, decreasing a blood total cholesterol level, decreasing a blood low-density lipoprotein, decreasing a blood very low-density lipoprotein level, improving leptin resistance, and/or improving insulin resistance, and combinations thereof.

In some embodiments, the benefit is achieved by browning of white adipose tissues or promoting activation of brown adipose tissues.

In some embodiments, the browning of white adipose tissues or promoting activation of brown adipose tissues is regulated by ADRB3 or β-AR.

In some embodiments, the benefit of the exercise is improved with administration of the composition relative to the same exercise regimen without administration of the composition.

In some embodiments, the subject is engaged in an exercise regimen. In some embodiments, the composition is administered to the subject prior to, during or after exercise or workout.

In some embodiments, the subject is human. In some embodiments, the subject is healthy human.

In some embodiments, the body weight was decreased about more than 10% with administration of GP combined with exercise relative to the same exercise without administration of GP. The TC was decreased about more than 30% or 40% with administration of GP combined with exercise relative to the same exercise without administration of GP. The food intake was reduced with administration of GP combined with exercise relative to the same exercise without administration of GP.

In some embodiments, the composition is administrated in an amount ranging from 2 mg/day-2000 mg/day. Preferably, it is administered at daily doses of 2 mg-1500 mg, 2 mg-1000 mg, 2 mg-800 mg, 2 mg-400 mg, 4 mg-1000 mg, 4 mg-700 mg, 4 mg-400 mg.

In some embodiments, the daily dose is administered in divided doses or a single dose.

In some embodiments, the administration is at least once a day or more times a day.

In some embodiments, the composition is administrated in a form of aqueous solution, aqueous suspension, capsule, drop, granule, liquid, powder, syrup, tablet, functionalized food, beverage, toothpaste, or sublingual articles.

In some embodiments, the composition is administrated orally, by intravenous injection, by intramuscular injection, intraperitoneally or sublingually.

In some embodiments, the composition is in an ingestible composition. The ingestible composition is selected from the group consisting of a bioceutical composition, a dietary supplement, a medicated feed, a nutraceutical composition.

In some preferred embodiments, the composition is prepared in a form of nutritional, or drinking, for use in a food, drink, nutritional products.

Another aspect of the present invention provides a composition for improving a benefit of an exercise experienced by a subject, wherein the composition comprises an effective amount of Grains of Paradise, analog or derivative thereof. In some embodiments, the benefit is achieved by browning of white adipose tissues or promoting activation of brown adipose tissues. In some embodiments, the browning of white adipose tissues or promoting activation of brown adipose tissues is regulated by ADRB3 or β-AR.

In some embodiments, the benefit comprises increasing energy expenditure, reducing a body fat percentage, reducing weight, lowering a blood glucose, decreasing a blood triglyceride level, decreasing a blood total cholesterol level, decreasing a blood low-density lipoprotein, decreasing a blood very low-density lipoprotein level, improving leptin resistance, and/or improving insulin resistance, and combinations thereof.

In some embodiments, the composition is administrated in an amount ranging from 2mg/day-2000 mg/day. Preferably, it is administered at daily doses of 2 mg-1500 mg, 2 mg-1000 mg, 2 mg-800 mg, 2 mg-400 mg, 4 mg-1000 mg, 4 mg-700 mg, 4 mg-400 mg.

In some embodiments, the daily dose is administered in divided doses or a single dose.

In some embodiments, the administration is at least once a day or more times a day.

In some embodiments, the composition is administrated in a form of aqueous solution, aqueous suspension, capsule, drop, granule, liquid, powder, syrup, tablet, functionalized food, beverage, toothpaste, or sublingual articles.

In some embodiments, the composition is prepared in a form of nutritional, or drinking, for use in a food, drink, nutritional products.

Another aspect of the present invention provides a use of Grains of Paradise, analog or derivative for preparing a composition for improving a benefit of an exercise experienced by a subject, wherein the composition comprises an effective amount of Grains of Paradise, an analog or derivative thereof. In some embodiments, the benefit is achieved by browning of white adipose tissues or promoting activation of brown adipose tissues. In some embodiments, the browning of white adipose tissues or promoting activation of brown adipose tissues is regulated by ADRB3 or β-AR.

In some embodiments, the benefit comprises increasing energy expenditure, reducing a body fat percentage, reducing weight, lowering a blood glucose, decreasing a blood triglyceride level, decreasing a blood total cholesterol level, decreasing a blood low-density lipoprotein, decreasing a blood very low-density lipoprotein level, improving leptin resistance, and/or improving insulin resistance, and combinations thereof.

In some embodiments, the subject is or is not engaged in an exercise regimen.

In some embodiments, the effect on body weight, TC, TG, LDL, VLDL of GP group is equivalent to or better than that of the exercise group.

In some embodiments, the benefit of the exercise is improved with administration of the composition relative to the same exercise regimen without administration of the composition.

In some embodiments, the composition is administrated in an amount ranging from 2mg/day-2000 mg/day. Preferably, it is administered at daily doses of 2 mg-1500 mg, 2 mg-1000 mg, 2 mg-800 mg, 2 mg-400 mg, 4 mg-1000 mg, 4 mg-700 mg, 4 mg-400 mg.

Another aspect of the present invention provides a method for providing a subject with a benefit associated with an exercise, comprising administration to the subject a composition comprising an effective amount of Grains of Paradise, analog or derivative thereof. In some embodiments, the benefit is achieved by browning of white adipose tissues or promoting activation of brown adipose tissues. In some embodiments, the browning of white adipose tissues or promoting activation of brown adipose tissues is regulated by ADRB3 or β-AR.

In some embodiments, the benefit comprises increasing energy expenditure, reducing a body fat percentage, reducing weight, lowering a blood glucose, decreasing a blood triglyceride level, decreasing a blood total cholesterol level, decreasing a blood low-density lipoprotein, decreasing a blood very low-density lipoprotein level, improving leptin resistance, and/or improving insulin resistance, and combinations thereof.

In some embodiments, the benefit of the exercise is improved with administration of the composition relative to the same exercise regimen without administration of the composition.

In some embodiments, the composition is administrated in an amount ranging from 2 mg/day-2000 mg/day.

In some embodiments, the composition is administrated in a form of aqueous solution, aqueous suspension, capsule, drop, granule, liquid, powder, syrup, tablet, functionalized food, beverage, toothpaste, or sublingual articles.

In some embodiments, the composition is prepared in a form of nutritional, or drinking, for use in a food, drink, nutritional.

### BRIEF DESCRIPTION OF THE FIGURES

FIG. 1 is a graph of energy expenditure change of GP group compared to control group at exercise and post exercise.

FIG. 2 is a graph of body weight of six groups of mice in 24 weeks.

FIG. 3 is a graph of fat mass of six groups of mice in 24 weeks.

FIG. 4 is a graph of TC level of six groups of mice.

FIG. 5 is a graph of TG level of six groups of mice.

FIG. 6 is a graph of LDL level of six groups of mice.

FIG. 7 is a graph of VLDL level of six groups of mice.

FIG. 8 is a graph of the food intake before and after leptin administration of six groups of mice.

FIG. 9 is a graph of Homeostasis model assessment index of six groups of mice.

FIG. 10 is a graph of glucose tolerance test results of six groups of mice.

FIG. 11 is a graph of insulin tolerance test results of six groups of mice.

FIG. 12 is a graph of ADRB3 and β-AR expression of six groups of mice.

FIG. 13 is a graph of energy expenditure results of six groups of mice.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

As used herein, the term “effective amount” refers to an amount that is required to achieve the effect as taught herein. It will be understood, however, that the total daily usage of Grain of Paradise or its analog or its derivatives may be decided by the attending physician within the scope of sound medical judgment. The specific therapeutically effective dose level for any particular subject will depend upon a variety of factors including the disorder being treated and the severity of the disorder; the specific composition employed, the age, body weight, general health, sex and diet of the subject; the time of administration, route of administration, and rate of excretion of Grain of Paradise or its analog or its derivatives employed; the duration of the treatment; drugs used in combination or coincidental with Grain of Paradise or its analog or its derivatives; and like factors well known in the medical arts. For example, it is well known within the skill of the art to start doses of the compound at levels lower than those required to achieve the desired therapeutic effect and to gradually increase the dosage until the desired effect is achieved.

One of skill in the art recognizes that an amount may be considered therapeutically “effective” even if the condition is not totally eradicated or prevented, but it or its symptoms and/or effects are improved or alleviated partially in the subject.

In some embodiments, the composition includes from about 5% to about 10%, 20%, 30%, 40%, 50%, 60%, 70%, 80%, 90%, or 95% by weight of Grain of Paradise or its analog or its derivatives, and preferably from about 30% to about 90% by weight of Grain of Paradise or its analog or its derivatives, based upon the total weight of the composition taken as 100% by weight.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. “subject” includes, without limitation, mice, rats, rabbits, guinea pigs, dogs, cats, sheep, goats, cows, horses, primates, such as monkeys, chimpanzees, and apes, and, in particular, humans.

Generally speaking, various embodiments of the present invention provide for improving a benefit of an exercise experienced by a subject, comprising administration to the subject a composition comprising an effective amount of Grains of Paradise, analog or derivative thereof. For instance, the benefit may include increasing energy expenditure, reducing a body fat percentage, reducing weight, lowering a blood glucose, decreasing a blood triglyceride level, decreasing a blood total cholesterol level, decreasing a blood low-density lipoprotein, decreasing a blood very low-density lipoprotein level, improving leptin resistance, and/or improving insulin resistance, and combinations thereof.

Grain of Paradise or its analog or its derivatives are administrated at a daily dose of 2 mg-2000 mg. The dosage may range broadly, depending upon the desired effects and the therapeutic indication. The daily dosage regimen for an adult human may be, for example, an oral dose of between 2 mg and 2000 mg of Grain of Paradise or its metabolites, preferably between 2 mg-1500 mg, 2 mg-1000 mg, 2 mg-800 mg, 2 mg-400 mg, 4 mg-1000 mg, 4 mg-700 mg, 4 mg-400 mg. Suitable dosage amounts for the compositions and methods of the disclosure range from about 1 to about 200 mg/kg/day, or from about 2 to about 150 mg/kg/day, or from about 2 to about 100 mg/kg/day, or from about 2 to about 80 mg/kg/day, or from about 4 to about 150 mg/kg/day, or from about 4 to about 80 mg/kg/day, suitable dosages for compositions of the present disclosure may be selected from 3 mg/kg/day,5 mg/kg/day, 10 mg/kg/day, 15 mg/kg/day, 20 mg/kg/day, 25 mg/kg/day, 30 mg/kg/day, 35 mg/kg/day, 40 mg/kg/day, 45 mg/kg/day, 50 mg/kg/day, 55 mg/kg/day, 60 mg/kg/day, 65 mg/kg/day, 70 mg/kg/day, 75 mg/kg/day, 80 mg/kg/day, 85 mg/kg/day, 90 mg/kg/day, 95 mg/kg/day, 100 mg/kg/day, 105 mg/kg/day, 110 mg/kg/day, 115 mg/kg/day, 120 mg/kg/day, 125 mg/kg/day, 130 mg/kg/day, 135 mg/kg/day, 140 mg/kg/day, 145 mg/kg/day, 150 mg/kg/day, 155 mg/kg/day, 160 mg/kg/day, 165 mg/kg/day, 170 mg/kg/day, 175 mg/kg/day, 180 mg/kg/day, 185 mg/kg/day, 190 mg/kg/day, and 195 mg/kg/day.

The dosage may be a single one or a series of two or more given in the course of one or more days, as is needed by the subject. In some embodiments, the compounds are administered for a period of continuous therapy, for example for a week or more, or for months or years. In some embodiments, Grain of Paradise or its metabolites, or a pharmaceutically acceptable salt thereof, can be administered less frequently compared to the frequency of administration of an agent within the standard of care. In some embodiments, Grain of Paradise or its metabolites, or a pharmaceutically acceptable salt thereof, can be administered one time per day. In some embodiments, the total time of the treatment regime with Grain of Paradise or its metabolites, or a pharmaceutically acceptable salt thereof, can be less compared to the total time of the treatment regime with the standard of care.

In some embodiments, the daily dose is administered in divided doses or a single dose. In some embodiments, the administration is at least once a day or more times a day. In some embodiments, the administration is at least 7 days and above in one period.

Compositions of the present disclosure may be provided in any suitable form and physical manifestation. By way of non-limiting example, the ingestible composition can be administered to a subject as a dietary supplement, a medicated feed, and a nutraceutical composition. Multiple techniques of administering a composition exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. By way of further non-limiting example, the compositions may be provided in any suitable physical form for oral administration, such as aqueous solutions or suspensions (e.g. an infused beverage, such as an energy beverage or energy “shot”), capsules (which may or may not be chewable), drops, granules, liquids, mists, powders, syrups, tablets (e.g. chewable, saliva-soluble, and/or swallowable tablets), functionalized foods (e.g. energy or nutrition bars, cookies, gums, candies, etc.), toothpastes, sublingual articles, and the like. In some embodiments, the composition may be provided in a form, e.g. a powder, that can be applied to a food (similar to a seasoning or condiment, etc.) or mixed with a beverage. Compositions of the present disclosure may thus comprise any suitable pharmaceutically acceptable additives, binders, and/or fillers, and may also comprise an active pharmaceutical or therapeutic agent other than Grain of Paradise.

The following examples are illustrative of select embodiments of the present invention and are not meant to limit the scope of the invention.

### EXAMPLES

### Example 1

25-30 years old healthy subjects are divided into GP group (n=3, 160 mg/day, Caloriburn, namely GP) and No supplement group (n=3, control). The calories burned at rest, exercise and post exercise (Rest 0-30 mins, Exercise 30-60 mins, recovery 60-90mins) in 3 healthy men are detected to see the effects of GP on calories. Calorie expenditure is measured 90 minutes after the subjects taking GP.

The clinical study is conducted with a randomized cross-over in which 3 healthy subjects (25-30 years old) gave informed consent to participate in the interventional study and were evaluated for their health and nutritional status, by anthropometric, and body composition evaluation. REE, TEE and metabolic equivalent are evaluated by SWA. For each subject we collect the following data: sex, age, weight and height, percentage of weight loss in the last six months, previous surgical treatment, smoking habit. In addition, BMI (Body Mass Index), REE (Resting energy expenditure), TEE (Total energy expenditure), MET (metabolic energy transformation), quality of life, muscle strength, body composition and daily dietary intake are evaluated. We also provide patients with a calorie equivalent diet to the TEE measured at baseline.

Evaluation of REE, TEE, MET are evaluated by the SenseWear Armband (SWA), a portable multi-sensor monitor already validated for the study of basal and total energy expenditure.

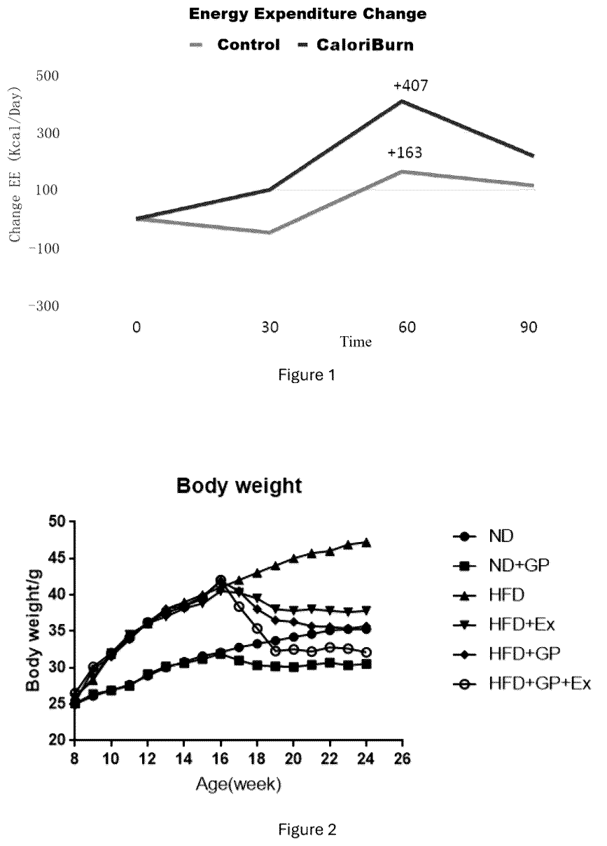

As shown in FIG. 1, GP increased the energy expenditure change compared to control group at exercise and post exercise, and GP increased the energy expenditure change by 249.69% compared to control group post exercise. The preliminary results indicated that GP may be beneficial in increasing calorie expenditure in this Pilot trial.

### Example 2

36 C57BL/6 male mice aged about 8 weeks, are randomly assigned to six study groups (GA1, GA2, GB1, GB2, GB3, GB4) of six mice each. Mice were maintained in a pathogen free condition under a strict 12 h light/dark cycle (0600/1800 h) with 12-15 cycles/hour of air change. Mice were provided with access to respective feed and water ad libitum as per experimental conditions. Mice were housed under controlled laboratory conditions of temperature and humidity at 23±2° C. and 50±10% RH, respectively.

Each study group consisted of six animals, and treatment details of the individual study groups are given below: Group A (GA1): Normal mice, maintained on normal diet for 8 weeks, receiving neither exercise nor Grain of Paradise treatment; Group A2 (GA2): Normal mice, maintained on a normal diet (ND) for 8 weeks, receiving only Grain of Paradise treatment and no exercise; Group B1 (GB1): Obese mice, maintained on high fat diet (HFD) for 8 weeks, receiving neither exercise nor Grain of Paradise treatment; Group B2 (GB2): Obese mice, maintained on HFD for 8 weeks, receiving only exercise and no Grain of Paradise treatment; Group B3 (GB3): Obese mice, maintained on HFD for 8 weeks, receiving only Grain of Paradise treatment and no exercise; Group B4 (GB4): Obese mice, maintained on HFD for 8 weeks, receiving Grain of Paradise treatment and exercise combination.

The total experimental time is 16 weeks, including two periods: an eight-week induction period and a subsequent eight-week treatment period. Throughout the study period, each mouse's body weight was measured weekly by a calibrated electronic digital weighing balance Sartorious BSA32025 (Sartorious AG, Gottingen, Germany).

At the end of the study, all mice were sacrificed using CO₂ suffocation, all relevant tissues were dissected, weighed, immediately snap frozen in liquid nitrogen, and stored at −80°C.

The synergism of Grain of Paradise administration could change the body composition.

Each animal's body composition was measured and recorded before the treatment and weekly during the study at 1200 h by Nuclear Magnetic Resonance Body Composition Analyzer in accordance with manufacturer's instruction.

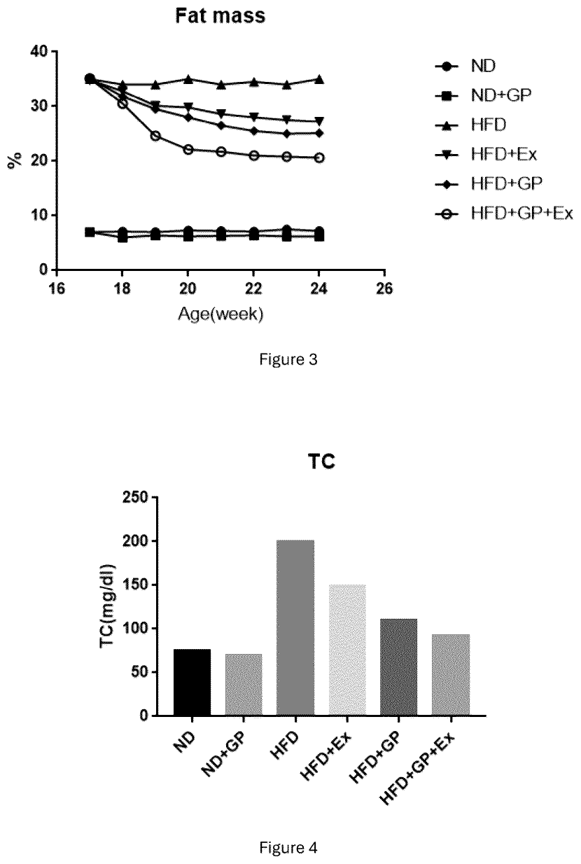

As shown in FIG. 2, during the 8-week induction period, the body weight of the 6 study groups of mice steadily increased. When the obese mice transferred to treatment, the mice treated with exercise or GP gradually lost weight to stabilize during treatment, and the effect on body weight of the GP group is equivalent to or better than that of the exercise group, while the mice treated with GP combined with exercise rapidly lost weight and reached a new stable baseline after 3 weeks. Body weight was 23.1% lower than before treatment. The body weight was decreased about more than 10% with administration of GP combined with exercise relative to the same exercise without administration of GP. In addition, as can be seen from FIG. 3, fat mass of mice in the HFD treatment group also decreased significantly with weight loss, especially in the exercise combined with GP treatment group.

The synergism of Grain of Paradise administration could improve biomarkers related to lipid mobilization in mice.

At the end of the study blood was collected from all test groups and various clinical parameters: plasma total cholesterol (TCHO), triglyceride (TG), low density lipoprotein (LDL), very low-density lipoprotein (VLDL), were measured using corresponding kits according to the manufacturers'instructions.

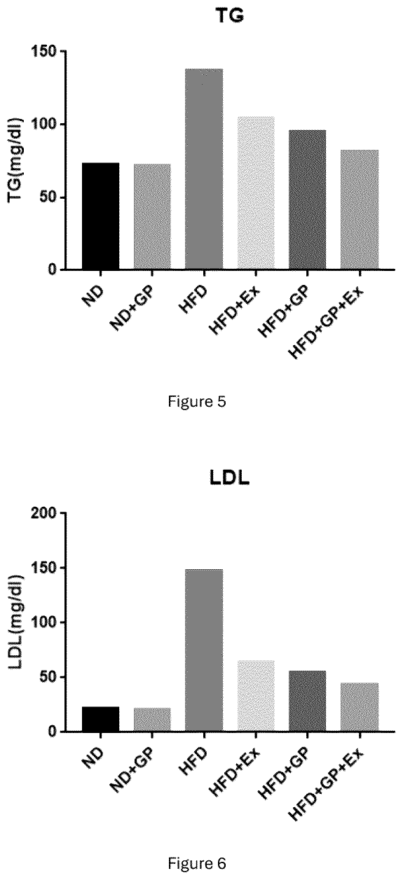

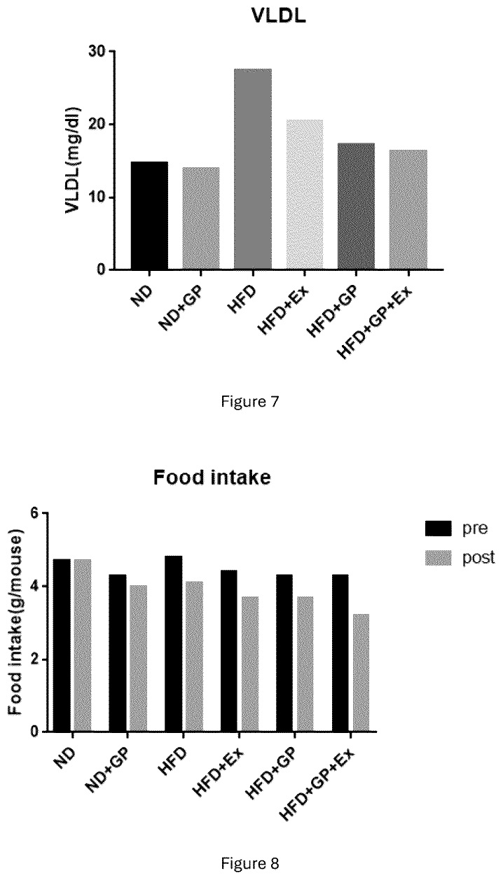

As can be seen from FIGS. 4 to 7, HFD causes significant increases in serum TC, TG, LDL, and VLDL. Exercise alone or treatment with Grain of Paradise alone resulted in significant reductions in TC, TG, LDL, and VLDL, and the effect on TC, TG, LDL, VLDL of the GP group is equivalent to or better than that of the exercise group. However, when the exercise and Grain of Paradise co-treatment, the reduction effect is more significant, TC content decreased by 54%, TG content decreased by 31.8%, LDL content decreased by 70.3%, VLDL content decreased by 40.3%. The TC was decreased about more than 30% or 40% with administration of GP combined with exercise relative to the same exercise without administration of GP.

The synergism of Grain of Paradise administration could improve leptin resistance and insulin resistance to ameliorate leptin and insulin sensitivity in mice.

Serum leptin resistance analysis. The leptin resistance was measured at the beginning and end of the treatment period. Mice were handled daily to minimize effects of stress on serum leptin measurements. Individual mice were transported into a procedure room for blood collection from a tail clip. Samples were allowed to coagulate for 30 min at room temperature and then were centrifuged at 2,500×g for 15 min at 4° C. Serum was collected and stored at −20°C for future analysis. To calculate leptin levels, a mouse leptin ELISA kit was used according to the manufacturer's protocol. Briefly, a reference curve was plotted using data from standards provided by the manufacturer, and the validity of the assay was confirmed using two separate quality control samples per assay. The measurements of unknown samples were then calculated using a four-parameter logistic function in Microsoft Excel.

Leptin sensitivity assay. The leptin sensitivity was measured at the beginning and end of the treatment period. Intraperitoneal injections of 50 μg of recombinant mouse leptin in 20 mM Tris, pH 8.0 were given just before initiation of the dark cycle. Food intake was assessed with a BioDAQ episodic intake monitor. Leptin sensitivity was assessed by comparing cumulative food intake for the dark cycle period after leptin injection with the cumulative food intake from the previous night when a control injection of 20 mM Tris, pH 8.0, was administered. The leptin sensitivity at each time point was carried out in each group of mice.

Assessment of leptin resistance in mice requires measurement of food intake. As shown in FIG. 8, we measured the food intake of mice one day before and after leptin administration. After intraperitoneal injection of leptin, the appetite of mice decreased and their food intake decreased significantly. Compared with the HFD group, the mice receiving the combination of exercise and GP reduced their food intake the most, which decreased by 25.6% compared with the previous day. The food intake was reduced with administration of GP combined with exercise relative to the same exercise without administration of GP.

Serum insulin resistance analysis. The insulin resistance was measured at the beginning and end of the treatment period. 12 h fasting blood samples were obtained for serum insulin and plasma glucose determinations in order to calculate the HOMA-IR. Blood samples for insulin were collected from the retro-orbital artery using a glass cannula and placed directly into an Eppendorf tube. It was then immediately placed in an ice-bath and centrifuged at 1000×g for 10 minutes at room temperature, according to the manufacturer's instructions. Serum was stored at −80° C. until the assay. Mice's insulin assay determinations were done by the luminescence method using relevant kits, with detection limit 28 pg/mL.

### The Synergism of Grain of Paradise Administration Could Reduce Blood Glucose and Insulin in Mice.

Glucose tolerance test (GTT): To measure glucose levels in blood, GTT were implemented in the mice (ND and HFD groups) after overnight by using a glucose analyzer in the 10th week of the study. The glucose concentrations were measured in blood collected by venous bleeding from the tail vein immediately at 0, 15, 30, 45, 60, 90 and 120 min after a bolus i.p. injection of glucose at 2 g/kg. Towards the end of the final week of the study, GTT was also performed on day 85 (day after the treatment period) for mice (ND and HFD groups).

Insulin tolerance test (ITT): To measure insulin levels in blood, ITT were measured in the 6-h fasting mice (ND and HFD groups) by using a glucose analyzer in the 11th week of the study. The glucose concentrations were measured by venous bleeding at 0, 15, 30, 45, 60, 90 and 120 min after an i.p. injection of human insulin at 0.75 U/kg. Towards the end of the final week of the study, ITT was performed on day 85 (day after the treatment period) for mice (ND and HFD groups).

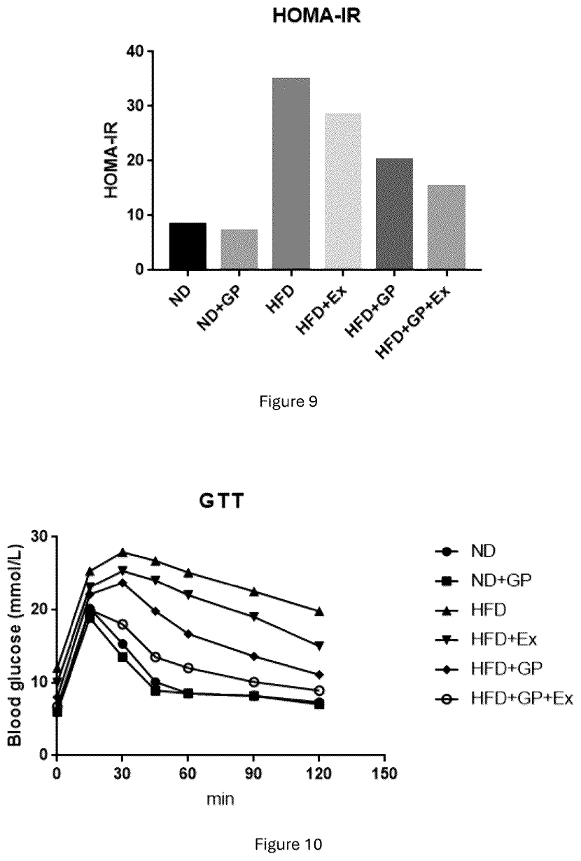

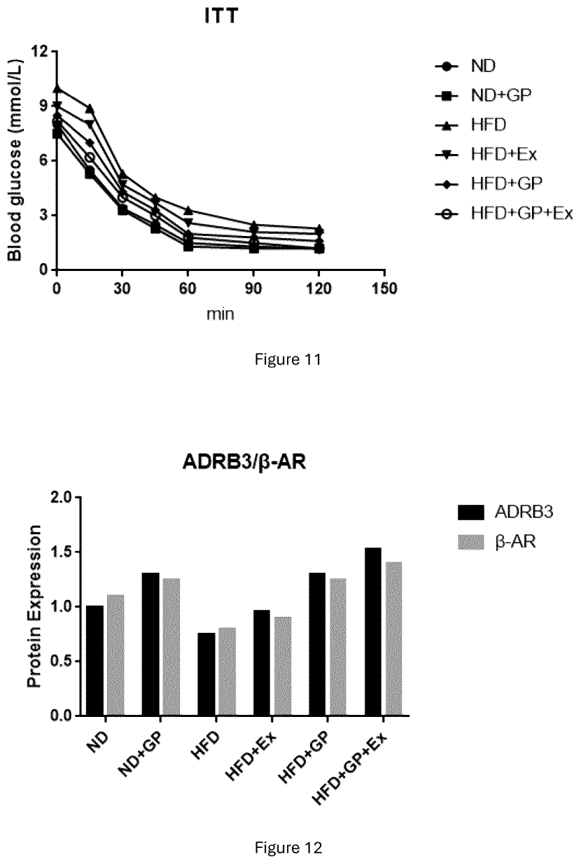

Compared with HFD control mice, the combination of exercise and Grain of Paradise treatment significantly improved glucose tolerance (FIG. 10) and showed higher insulin sensitivity (FIG. 11). Homeostasis model assessment index (HOMA-IR) of insulin resistance was increased in HFD control mice (FIG. 9), while HOMA-IR index values were significantly decreased in cooperatively treated HFD mice.

The synergism of Grain of Paradise administration could brown of white adipose tissues or promote activation of brown adipose tissues mediated by regulating the abundance of the ADRB3 or β-AR in mice.

The levels of ADRB3 and β-AR protein from brown adipose tissues were determined by western blot analysis.

Activating nonshivering thermogenesis in brown adipose tissue (BAT) is a promising strategy to prevent obesity. ADRB3 gene is a major regulator of norepinephrine-induced nonshivering thermogenesis. This study investigated whether the Grain of Paradise could synergize with exercise to increase the non-shivering thermogenesis of BAT and white adipose tissue (WAT) in mice to improve obesity. As shown in FIG. 12, the expression of ADRB3 and β-AR protein in HFD-treated mice was 0.75 and 0.8, compared with this, the expression of the ADRB3 protein was increased in the BAT of the mice of exercise or Grain of Paradise treatment. Moreover, when the exercise mice were treated with Grain of Paradise at the same time, the expression of ADRB3 and β-AR protein were significantly increased to 1.53 and 1.4.

### The Synergism of Grain of Paradise Administration Could Increase the Calorie Expenditure in Mice

Indirect calorimetry chambers (Oxymax/CLAMS; Columbus Instruments) were used to measure energy expenditure through detection of O₂ consumption and CO₂ production within each chamber (22° C., 2.5 L volume, flow rate: 0.5 L/min, sampling flow: 0.4/min) in 13 min bins.

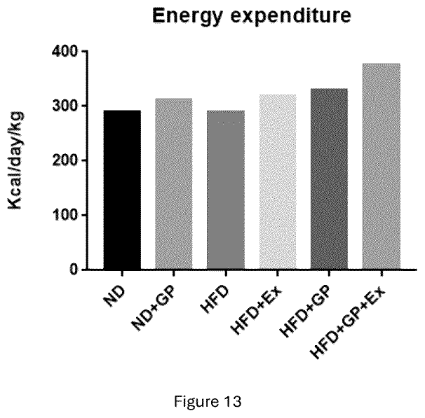

As shown in FIG. 13, compared to mice fed only HFD, the energy expenditure of mice subjected to exercise treatment was increased by 10.3%, while the energy expenditure of mice subjected to GP treatment was increased by 13.4%. The energy expenditure of mice subjected to both exercise and GP treatment was significantly increased by 29.3%.

Serum FGF21 assay and UCP1

Serum fibroblast growth factor 21 (FGF21) were assessed in a 96-well microplate using a mouse/rat FGF21 quantikine ELISA kit, and read at 450 nm with a microplate reader.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

It is to be understood that while the invention has been described in conjunction with the detailed description thereof and accompanying figures, the foregoing description and accompanying figures are only intended to illustrate, and not limit the scope of the invention, which is defined by the scope of the appended claims. Other aspects, advantages, and modifications are within the scope of the following claims. All publications referenced herein are incorporated by reference in their entireties.

What is claimed is:

1. A method for improving a benefit of an exercise experienced by a subject or providing a subject with a benefit associated with an exercise, comprising administration to the subject a composition comprising an effective amount of Grains of Paradise, analog or derivative thereof.

2. The method of claim 1, wherein the benefit comprises increasing energy expenditure, reducing a body fat percentage, reducing weight, lowering a blood glucose, decreasing a blood triglyceride level, decreasing a blood total cholesterol level, decreasing a blood low-density lipoprotein, decreasing a blood very low-density lipoprotein level, improving leptin resistance, and/or improving insulin resistance, and combinations thereof.

3. The method of claim 1, wherein the benefit is achieved by browning of white adipose tissues or promoting activation of brown adipose tissues.

4. The method of claim 3, wherein the browning of white adipose tissues or promoting activation of brown adipose tissues is regulated by ADRB3 or β-AR.

5. The method of claim 1, wherein the benefit of the exercise is improved with administration of the composition relative to the same exercise regimen without administration of the composition.

6. The method of claim 1, wherein the composition is administrated in an amount ranging from 2mg/day-2000 mg/day.

7. The method of claim 1, wherein the composition is administrated in a form of aqueous solution, aqueous suspension, capsule, drop, granule, liquid, powder, syrup, tablet, functionalized food, beverage, toothpaste, or sublingual articles.

8. The method of claim 1, wherein the composition is in an ingestible composition, wherein the ingestible composition is selected from the group consisting of a bioceutical composition, a dietary supplement, a medicated feed, a nutraceutical composition.

9. The method of claim 1, wherein the composition is prepared in a form of nutritional, or drinking, for use in a food, drink, nutritional products.

10. The method of claim 1, wherein the composition is administered to the subject prior to, during or after exercise or workout.

11. The method of claim 1, wherein, wherein the subject is human.

12. The method of claim 1, wherein, wherein the subject is healthy human.
